(19) United States
(12) Patent Application Publication
(10) Pub. No.: US 2023/0241396 A1
(43) Pub. Date: Aug. 3, 2023

(54) AN IMPLANTABLE NEUROMODULATION SYSTEM UTILISING CLOSED LOOP CONTROL

(71) Applicant: GALVANI BIOELECTRONICS LIMITED, Brentford, Middlesex (GB)

(72) Inventors: Michael John CARR, Brentford, Middlesex (GB); Rizwan BASHIRULLAH, Brentford, Middlesex (GB)

(73) Assignee: GALVANI BIOELECTRONICS LIMITED, Brentford, Middlesex (GB)

(21) Appl. No.: 18/002,179

(22) PCT Filed: Jun. 18, 2021
(86) PCT No.: PCT/GB2021/051553

§ 371 Date: Dec. 16, 2022

Related U.S. Application Data
(60) Provisional application No. 63/041,453, filed on Jun. 19, 2020.

(51) Int. Cl. A61N 1/36; A61N 1/05

(52) U.S. Cl. CPC A61N 1/36139; A61N 1/36153; A61N 1/0556; A61N 1/36053

(57) ABSTRACT

The present invention provides an implantable neuromodulation system for delivering an electrical signal to a nerve to stimulate, inhibit or block conduction of action potentials in the nerve. The system comprises a neural interface device comprising first and second electrodes; a signal generator and a first closed-loop controller configured to generate a control signal based a property of the signal based on a measured voltage across the first and second electrodes, and cause the signal generator to adjust the electrical signal to modify the property of the signal.

### TECHNICAL FIELD

Described herein is closed-loop control of a system designed to stimulate or inhibit, preferably block conduction of action potentials in neurons. Embodiments of the disclosure include a system to generate and deliver electrical signal to electrodes within one or more nerve cuffs attached to a nerve trunk. The applied signal overrides the activity that would otherwise be conducted along the nerve. Application of the signal results in a stimulation or inhibition, preferably block of neuronal activity.

### SUMMARY

In a first aspect, the present disclosure provides an implantable neuromodulation system for delivering an electrical signal, preferably an LFAC electrical signal, to a nerve to stimulate or inhibit, optionally block, conduction of action potentials in the nerve, the system comprising: a neural interface device for stimulating or inhibiting, optionally blocking, neural activity in the nerve, the at least one neural interface device comprising first and second electrodes; a signal generator electrically coupled to the first and second electrodes and configured to generate the electrical signal that, when applied to the nerve via the first and second electrodes, stimulates or inhibits, optionally blocks, neural activity in the nerve; a first closed-loop controller comprising a sensor coupled between the first and second electrodes and configured to determine a voltage across the first and second electrodes when the electrical signal is applied to the nerve via the first and second electrodes, the first closed-loop controller further comprising a processor to determine a property of the signal based on the measured voltage; wherein the first closed-loop controller is further configured to generate a control signal based on the determined property, the control signal configured to cause the signal generator to adjust the electrical signal to modify the property of the signal.

One advantage of a closed-loop control system to control voltage off-set of the signal across the electrodes is that nerve injuring and/or corrosive reactions at the electrode surface can be prevented by actively controlling the voltage-off set across the electrodes. Such control also allows active control of the applied current used to compensate for the off-set that would otherwise lead to an undesirable voltage off-set.

Optionally, the determined property is a voltage offset between the first and second electrodes, and wherein the control signal is configured to cause the signal generator to adjust the electrical signal to reduce the voltage offset, preferably toward 0 v, more preferably to 0 v.

Optionally, the determined property is a magnitude of the voltage across the first and second electrodes, and wherein the control signal is configured to cause the signal generator to adjust the electrical signal to reduce the magnitude of the voltage.

Optionally, the processor is further configured to compare the determined magnitude of the voltage across the first and second electrodes with a predetermined threshold, and generate the control signal based on the comparison.

Optionally, the predetermined threshold is an upper voltage limit, preferably a safe voltage limit, more preferably a safe voltage limit based upon the material of the first and second electrodes.

Optionally, the control signal is configured to cause the signal generator to increase or decrease the current (I_{P}, I_{N}) of the electrical signal, preferably between a range having a lower limit of 100 μA and an upper limit of 1 mA, preferably 500 μA, preferably 200 μA.

Optionally, the system further comprising a second closed-loop controller comprising a physiological sensor configured to measure a physiological parameter of a patient when the electrical signal is applied to the nerve via the first and second electrodes, wherein the physiological parameter is affected by the stimulation or inhibition, optionally block, of the neural activity in the nerve, the second closed-loop controller further comprising a processor to determine a deviation from a target physiological parameter, based on the measured physiological parameter; wherein the second closed-loop controller is further configured to generate a control signal based on the deviation, the control signal configured to cause the signal generator to adjust the LFAC electrical signal to reduce the deviation from the target physiological parameter.

In a second aspect, the present disclosure provides an implantable neuromodulation system for delivering an electrical signal, preferably an LFAC electrical signal, to a nerve to stimulate or inhibit, optionally block, conduction of action potentials in the nerve, the system comprising: a neural interface device for stimulating or inhibiting, optionally blocking, neural activity in the nerve, the at least one neural interface device comprising first and second electrodes; a signal generator electrically coupled to the first and second electrodes and configured to generate the electrical signal that, when applied to the nerve via the first and second electrodes, stimulates or inhibits, optionally blocks, neural activity in the nerve; a first closed-loop controller comprising a physiological sensor configured to measure a physiological parameter of a patient when the electrical signal is applied to the nerve via the first and second electrodes, wherein the physiological parameter is affected by the stimulation or inhibition, optionally blocking, of the neural activity in the nerve, the first closed-loop controller further comprising a processor to determine a deviation from a target physiological parameter, based on the measured physiological parameter; wherein the first closed-loop controller is further configured to generate a control signal based on the determined deviation, the control signal configured to cause the signal generator to adjust the electrical signal to reduce the deviation from the target physiological parameter.

One advantage of a closed-loop control system to control the magnitude of the voltage developed across the electrodes during the application of a blocking signal is to control the range of undesired electrochemical reaction that could occur at the nerve electrode interface, for example to prevent hydrolysis of water and prevent corrosive reactions of the electrode material, or undesired redox reactions in the media surrounding the electrode, i.e. biological tissue or electrolyte. A further advantage is that the systems target voltage can be selected depending on the electrode material of the interface.

Optionally, the control signal is configured to cause the signal generator to increase or decrease the current (I_{P}, I_{N}) of the LFAC electrical signal. Further optionally, the control signal is configured to cause the signal generator to increase or decrease the current (I_{P}, I_{N}) of the LFAC electrical signal between a range having a lower limit of 100 μA and an upper limit of 1 mA, preferably 500 μA, preferably 200 μA.

Optionally, the control signal is configured to cause the signal generator to increase or decrease the frequency of the electrical signal, preferably between a range having a lower limit of 0.1 Hz and an upper limit of 100 Hz.

Optionally, the physiological sensor is one or more of a heart rate sensor, respiratory rate sensor, pulse oximetry sensor, blood pressure sensor, blood glucose sensor, and ECG (electrocardiogram) sensor, bladder pressure sensor and physiological parameter is one or more of cardiac function, respiratory function, ECS, heart rate, respiratory rate, pulse oximetry, bladder pressure, blood pressure and blood glucose.

Optionally, the system further comprises a second closed-loop controller comprising a sensor coupled between the first and second electrodes and configured to determine a voltage across the first and second electrodes when the electrical signal is applied to the nerve via the first and second electrodes, the second closed-loop controller further comprising a processor to determine a property of the signal based on the measured voltage; wherein the second closed-loop controller is further configured to generate a control signal based on the determined property, the control signal configured to cause the signal generator to adjust the electrical signal to modify the property of the signal.

Optionally, the system comprises a third electrode, preferably having a larger surface area than the first and second electrodes.

Optionally, the implantable neuromodulation system is enclosed in a housing, and wherein the third electrode is formed from at least part of, or preferably all of the housing.

In a third aspect, the present disclosure provides an implantable neuromodulation system for delivering electrical signals, preferably LFAC electrical signal, to a nerve to stimulate or inhibit, optionally block, conduction of action potentials in the nerve, the system comprising: a neural interface device for stimulating or inhibiting, optionally blocking, neural activity in the nerve, the at least one neural interface device comprising at least first and second pairs of electrodes; a signal generator electrically coupled to the at least first and second pairs of electrodes and configured to generate first and second electrical signals that, when applied to the nerve via the first and second pairs of electrodes respectively, stimulates or inhibits, optionally blocks, neural activity in the nerve; wherein the first electrical signal applied to the first pair of electrodes is out of phase with the second electrical signal applied to the second pair of electrodes.

Optionally, the first pair of electrodes comprises first and second electrodes, and the second pair of electrodes comprises third and fourth electrodes.

Optionally, the first and second electrical signals are configured such that, when applied to the nerve via the first and second pairs of electrodes respectively, the first electrical signal stimulates or inhibits, optionally blocks, neural activity in the nerve for a first period and the second electrical signal stimulates or inhibits, optionally blocks, neural activity in the nerve for a second period following the first period.

Optionally, the first and second electrical signals are further configured such that, when applied to the nerve via the first and second pairs of electrodes respectively, the first electrical signal stimulates or inhibits, optionally blocks, neural activity in the nerve for a third period following the second period, and the second electrical signal stimulates or inhibits, optionally blocks, neural activity in the nerve for a fourth period following the third period.

Optionally, each period corresponds to a 90° phase of the first and second electrical signals.

Optionally, the first and second electrical signals are configured such that, when applied to the nerve via the first and second pairs of electrodes respectively, the first electrical signal stimulates or inhibits, optionally blocks, neural activity in the nerve in an upstream direction from stimulation by the second electrical signal, and the second electrical signal stimulates or inhibits, optionally blocks, neural activity in the nerve in a downstream direction from stimulation by the first electrical signal.

Optionally, the first electrical signal is out of phase with the second electrical signal by between 1° and 180°, preferably by between 45° and 135°, more by preferably by between 80° and 100°, most preferably by 90°.

In a fourth aspect, the present disclosure provides an implantable neuromodulation system for delivering electrical signals to a nerve to stimulate or inhibit, optionally block, conduction of action potentials in the nerve, the system comprising: a neural interface device for stimulating or inhibiting, optionally blocking, neural activity in the nerve, the at least one neural interface device comprising at least first and second pairs of electrodes; a signal generator electrically coupled to the at least first and second pairs of electrodes and configured to generate an inhibition electrical signal, preferably an LFAC electrical signal, that, when applied to the nerve via the first pair of the electrodes, inhibits, optionally blocks, neural activity in the nerve; the signal generator further configured to generate a stimulation signal that, when applied to the nerve via the second pair of the electrodes, stimulates neural activity in the nerve; wherein the signal generator is configured to apply the stimulation signal to the nerve via the second pair of electrodes during a blocking window of the inhibition electrical signal applied to the nerve via the first pair of electrodes such that the inhibition electrical signal applied to the nerve via the first pair of electrodes inhibits, optionally blocks, propagation of the stimulation signal applied to the nerve via the second pair of electrodes.

For the avoidance of doubt, a ‘pair’ of electrodes should not be interpreted in such a way that first and second ‘pairs’ of electrodes requires four independent electrodes. In certain embodiments, a given electrode may belong to more than one pair. For example, a set of three electrodes may provide first and second pairs whereby one of the three electrodes belonging to the first and second pairs.

Optionally, wherein the blocking window is within a predetermined threshold phase difference from a peak and/or trough of the inhibition electrical signal.

Optionally, wherein the predetermined threshold phase difference is ±90°, preferably ±70°, preferably ±55°, preferably ±45°, preferably ±35°, preferably ±25°, preferably ±15°, preferably ±10°, preferably ±5°, preferably ±2°, preferably ±1° of the peak and/or trough of the inhibition electrical signal.

Optionally, the neural interface device comprises at least first, second and third electrodes, and wherein the first pair of electrodes comprises the first and second electrodes, and the second pair of electrodes comprises the second and third electrodes.

Optionally, the neural interface device comprises at least first, second, third and fourth electrodes, and wherein the first pair of electrodes comprises the first and second electrodes, and the second pair of electrodes comprises the third and fourth electrodes.

Optionally, the neural interface device is configured in use to extend along a nerve from a first end of the device to a second end, and wherein the first and second pairs of electrodes are spaced apart between the first and second ends such that at least one electrode of the first pair of electrodes is proximate the first end and at least one electrode of the second pair of electrodes is proximate the second end.

Optionally the first electrode is proximate the first end, the third electrode is proximate the second end and the second electrode is between the first and third electrodes.

Optionally the first and second electrodes are proximate the first end, and the third and fourth electrodes are proximate the second end.

Optionally the signal generator is configured to apply the stimulation signal to the nerve via the second pair of electrodes when the inhibition electrical signal applied to the nerve via the first pair of electrodes is at a trough of the inhibition electrical signal at the second electrode.

Optionally, with respect to the third and fourth aspects, the system further comprises: a physiological closed-loop controller comprising a physiological sensor configured to measure a physiological parameter of a patient when the electrical signal is applied to the nerve via the first and second electrodes, wherein the physiological parameter is affected by the stimulation or inhibition, optionally blocking, of the neural activity in the nerve, the physiological closed-loop controller further comprising a processor to determine a deviation from a target physiological parameter, based on the measured physiological parameter; wherein the physiological closed-loop controller is further configured to generate a control signal based on the determined deviation, the control signal configured to cause the signal generator to adjust the electrical signal to reduce the deviation from the target physiological parameter. Further optionally, the electrical signal is an LFAC signal or an inhibition signal.

Optionally, the system further comprising a signal property closed-loop controller comprising a sensor coupled between the first and second electrodes and configured to determine a voltage across the first and second electrodes when the electrical signal is applied to the nerve via the first and second electrodes, the signal property closed-loop controller further comprising a processor to determine a property of the signal based on the measured voltage; wherein the signal property closed-loop controller is further configured to generate a control signal based on the determined property, the control signal configured to cause the signal generator to adjust the electrical signal to modify the property of the signal. Further optionally, the electrical signal is an LFAC signal or an inhibition signal.

Optionally, the implantable neuromodulation system is enclosed in a housing, and the implantable neuromodulation system further comprises a reference electrode. Further optionally, the wherein the reference electrode has a larger surface area than the other electrodes. Further optionally, the reference electrode is formed from at least part of or optionally all of the housing.

### BRIEF DESCRIPTION OF THE DRAWINGS

Embodiments of the disclosure will now be described in detailed with reference to the accompanying drawings, in which:

FIG. 1 shows a diagrammatic representation of a first embodiment of an implantable neuromodulation system according to the invention including a feedback loop for sensing voltage across first and second electrodes.

FIG. 2 shows a diagrammatic representation of a second embodiment of an implantable neuromodulation system according to the invention include a feedback loop for sensing physiological parameters.

FIG. 3 shows a diagrammatic representation of a third embodiment of an implantable neuromodulation system according to the invention comprising three electrodes arranged in two pairs.

FIG. 4 shows a diagrammatic representation of a fourth embodiment of an implantable neuromodulation system according to the invention comprising four electrodes arranged in two pairs.

FIG. 5 shows a diagrammatic representation of the third embodiment of the invention, further comprising the feedback loop for sensing voltage across the electrodes as shown in FIG. 1.

FIG. 6 shows a diagrammatic representation of the fourth embodiment of the invention, further comprising the feedback loop for sensing voltage across the electrodes as shown in FIG. 1.

FIG. 7 shows a diagrammatic representation of the third embodiment of the invention, further comprising the feedback loop for sensing physiological parameters as shown in FIG. 2.

FIG. 8 shows a diagrammatic representation of the fourth embodiment of the invention, further comprising the feedback loop for sensing physiological parameters as shown in FIG. 2.

FIG. 9 shows an alternative diagrammatic representation of the first embodiment of the disclosure.

FIG. 10 shows a current response of the system shown in FIG. 9.

FIG. 11 shows an alternative diagrammatic representation of the first embodiment of the invention further comprising the feedback loop for sensing physiological parameters as shown in FIG. 2.

FIG. 12 shows an alternative diagrammatic representation of the fourth embodiment of the invention further comprising the feedback loops for sensing voltage across the electrodes as shown in FIG. 6 and for sensing physiological parameters as shown in FIG. 8.

FIG. 13 shows an alternative configuration of the embodiment shown in FIG. 12.

FIG. 14 shows an alternative diagrammatic representation of the third embodiment of the invention further comprising the feedback loops for sensing voltage across the electrodes as shown in FIG. 5 and for sensing physiological parameters as shown in FIG. 7.

### DETAILED DESCRIPTION

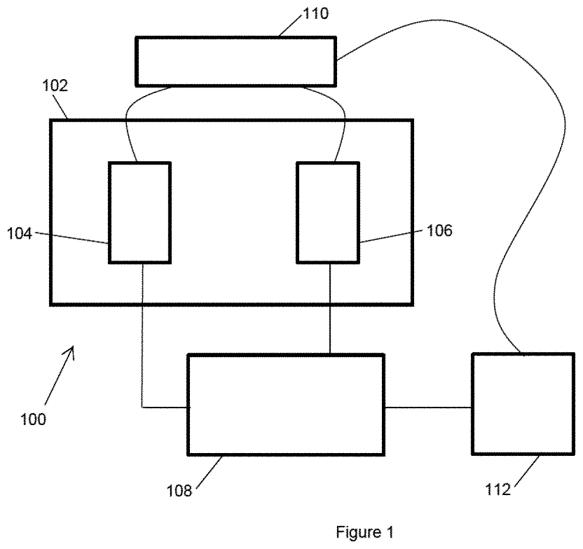

As will now be described, FIGS. 1 to 8 show eight alternative embodiments with which the aspects of the present disclosure may be implemented. The structure and components of these embodiments will now be described with reference to the drawings. A more detailed explanation of the operation and function of the embodiments in the context of the disclosure will follow on.

FIG. 1 shows a diagram of a first embodiment of an implantable neuromodulation system 100 according to the disclosure.

The neuromodulation system 100 comprises a neural interface device 102 for stimulating and/or inhibiting, preferably blocking neural activity in a nerve such as the cervical vagus nerve (not shown). Example neural interface embodiments may comprise of neural cuffs that fully or partially circumferentially enclose a segment of the nerve, flat neural interface such as patch electrodes, probe electrodes, or other types of extravascular neural interface, intravascular neural interface, or intrafascicular neural interface. The system may be used on any nerve that produces a physiological response when suitably stimulated. Examples include, but not limited to the cervical vagus nerve (which may produce an increase or decrease in heart rate), the splenic nerve (which may produce an increase or decrease in blood pressure), the carotid sinus nerve, the sympathetic chain, the renal nerve, the superior ovarian nerve, paravertebral chain (T1-T2 segment) and GSN (greater splanchnic nerve).

For example, the neuromodulation system described herein can be suitable for any therapeutic indication that uses LFAC signal or involves inhibiting, optionally blocking, neural signals. For example, the neuromodulation system can be used for, but not limited to, treating hypertension, cardiac arrhythmia, ventricular tachyarrhythmia, type 2 diabetes or polycystic ovary syndrome.

In other embodiments two or more neural interface devices may be provided, and any plurality of such neural interface devices may be separate or coupled. The neural interface devices may be in the form of a cuff, or any other interface suitable for attaching to or being positioned adjacent a nerve, a nerve bundle, a vessel, and/or an organ. For example, the neural interface embodiments may comprise of neural cuffs that fully or partially circumferentially enclose a segment of the nerve, flat surface neural interface such as patch electrodes, probe electrodes, or other types of extravascular neural interface, intravascular neural interface, or intrafascicular neural interface.

The neural interface device 102 comprises first and second electrodes 104, 106. Where two or more neural interface devices are provided, each may have one or more electrodes. For instance, a system may comprise first and second neural interface devices, wherein the first neural interface device comprises a first electrode of the system, and the second neural interface device comprises a second electrode of the system. In some embodiments described in more detail elsewhere herein, the ‘first electrode’ may be a pair of ‘first electrodes’ such that a bipolar signal can be applied across the electrodes in the pair. Likewise, the ‘second electrode’ may be a pair of ‘second electrodes’ such that a bipolar signal can be applied across the electrodes in the pair. Thus, in one embodiment, the neural interface device may comprise four electrodes; i.e. two pairs. In another embodiment described in more detail elsewhere herein, the first and second electrodes may share a common third electrode which is again used to apply bipolar signals between the first electrode and the common third electrode, and between the second electrode and the common third electrode. In another embodiment, first and second electrodes are used and the signals are monopolar.

The neuromodulation system 100 comprises signal generator 108 electrically coupled to the first and second electrodes 104, 106. In some embodiments of the invention the signal generator is implanted together with other components of the implantable system, whilst in other embodiments of the disclosure the signal generator may be implanted at a different location to the other components of the implantable system, whilst in still other embodiments of the invention, the signal generator may be external to the patient. The signal generator 108 is configured to generate a low frequency alternating current (LFAC) signal described elsewhere herein, which it applies to the nerve to which the neural interface device 102 is attached via the first and second electrodes 104, 106.

The neuromodulation system 100 further comprises a voltage sensor 110 coupled between the first and second electrode 104, 106. The voltage sensor 110 is configured to measure a voltage across the first and second electrodes whilst the signal generator 108 applies the LFAC signal to the nerve to which the neural interface device 102 is attached via the first and second electrodes 104, 106.

The neuromodulation system 100 further comprises a control system 112 communicatively coupled to the voltage sensor 110. The function of the control system 112 is to generate a feedback response upon receiving a signal from the voltage sensor.

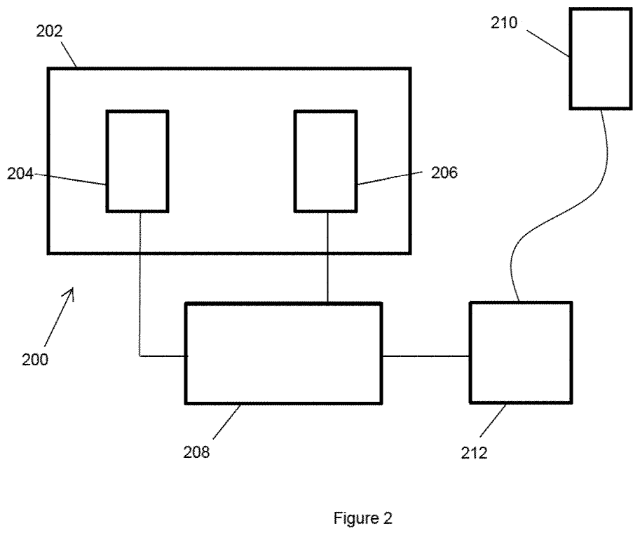

FIG. 2 shows a diagram of a second embodiment of an implantable neuromodulation system 200 according to the disclosure.

The neuromodulation system 200 comprises a neural interface device 202 for stimulating and/or inhibiting, preferably blocking neural activity in a nerve such as the cervical vagus nerve (not shown). Example neural interface embodiments may comprise of neural cuffs that fully or partially circumferentially enclose a segment of the nerve. The system may be used on any nerve that produces a physiological response when suitably stimulated. Examples include the cervical vagus nerve (which may produce an increase or decrease in heart rate) and the splenic nerve (which may produce an increase or decrease in blood pressure).

In other embodiments two or more neural interface devices may be provided, and any plurality of such neural interface devices may be separate or coupled. The neural interface devices may be in the form of a cuff, or any other interface suitable for attaching to or being positioned adjacent a nerve.

The neural interface device 202 comprises first and second electrodes 204, 206. Where two or more neural interface devices are provided, each may have one or more electrodes. For instance, a system may comprise first and second neural interface devices, wherein the first neural interface device comprises a first electrode of the system, and the second neural interface device comprises a second electrode of the system. In some embodiments described in more detail elsewhere herein, the ‘first electrode’ may be a pair of ‘first electrodes’ such that a bipolar signal can be applied across the electrodes in the pair. Likewise, the ‘second electrode’ may be a pair of ‘second electrodes’ such that a bipolar signal can be applied across the electrodes in the pair. Thus, in one embodiment, the neural interface device may comprise four electrodes; i.e. two pairs. In another embodiment described in more detail elsewhere herein, the first and second electrodes may share a common third electrode which is again used to apply bipolar signals between the first electrode and the common third electrode, and between the second electrode and the common third electrode. In another embodiment, first and second electrodes are used and the signals are monopolar.

The neuromodulation system 200 comprises signal generator 208 electrically coupled to the first and second electrodes 204, 206. The signal generator 208 is configured to generate a low frequency alternating current (LFAC) signal described elsewhere herein, which it applies to the nerve to which the neural interface device 202 is attached via the first and second electrodes 204, 206.

The neuromodulation system 200 further comprises a physiological sensor 210 to detect the at least one pre-determined physiological response. In some cases, a plurality of such sensors may be used, which may sense the same or different physiological responses. Exemplary sensors include a heart rate sensor, a blood pressure sensor a pulse oximetry sensor, a sensor for detecting cardiac function and a sensor for detecting respiratory function.

The neuromodulation system 200 further comprises a control system 212 communicatively coupled to the physiological sensor 210. The function of the control system 212 is to generate a feedback response upon receiving a signal from the physiological sensor.

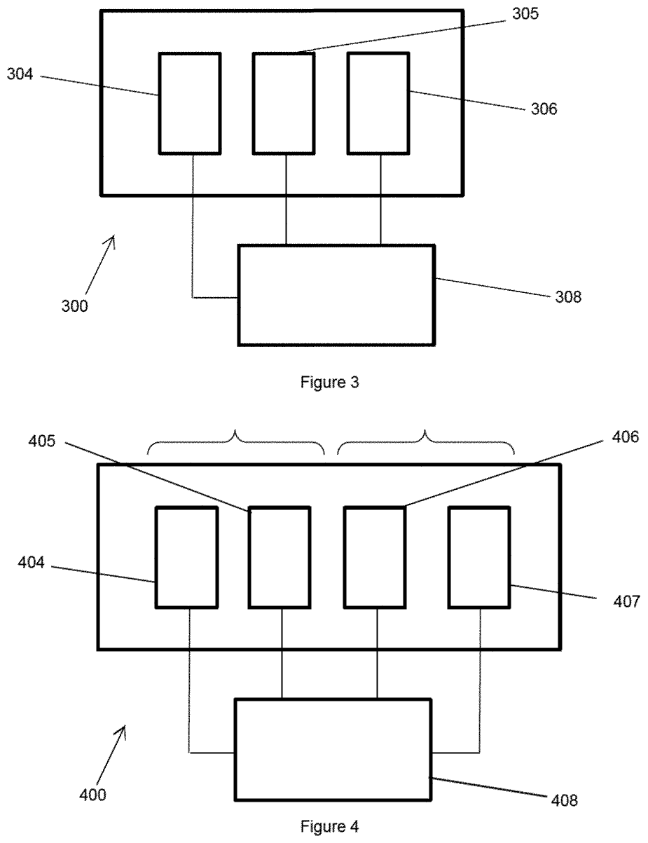

FIG. 3 shows a diagram of a third embodiment of an implantable neuromodulation system 300 according to the disclosure.

The neuromodulation system 300 comprises a neural interface device 302 for stimulating and/or inhibiting, preferably blocking neural activity in a nerve such as the cervical vagus nerve (not shown). Example neural interface embodiments may comprise of neural cuffs that fully or partially circumferentially enclose a segment of the nerve. The system may be used on any nerve that produces a physiological response when suitably stimulated. Examples include the cervical vagus nerve (which may produce an increase or decrease in heart rate) and the splenic nerve (which may produce an increase or decrease in blood pressure).

In other embodiments two or more neural interface devices may be provided, and any plurality of such neural interface devices may be separate or coupled. The neural interface devices may be in the form of a cuff, or any other interface suitable for attaching to or being positioned adjacent a nerve.

The neural interface device 302 comprises first, second and third electrodes 304, 305, 306. Where two or more neural interface devices are provided, each may have one or more electrodes. For instance, a system may comprise first and second neural interface devices, wherein the first neural interface device comprises a first electrode of the system, and the second neural interface device comprises a second and third electrode of the system. Alternatively, a system may comprise first, second and third neural interface devices, wherein the first neural interface device comprises a first electrode of the system, the second neural interface device comprises a second electrode of the system and the third neural interface device comprises a third electrode of the system.

The neuromodulation system 300 comprises signal generator 308 electrically coupled to the first, second and third electrodes 304, 305, 306. The signal generator 308 is configured to generate a low frequency alternating current (LFAC) signal described elsewhere herein, which it applies to the nerve to which the neural interface device 302 is attached via the first, second and third electrodes 304, 305, 306.

In the case of the third embodiment, the signal generator is configured to generate first and second LFAC electrical signals, which it applies to the nerve to which the neural interface device 302 is attached via two corresponding pairs of the first, second and third electrodes 304, 305, 306. For example, the signal generator may apply the first LFAC electrical signal to the nerve by applying a signal between the first and second electrodes 304, 305 and may apply the second LFAC electrical signal to the nerve by applying a signal between the second and third electrodes 305, 306.

FIG. 4 shows a diagram of a fourth embodiment of an implantable neuromodulation system 400 according to the disclosure.

The neuromodulation system 400 comprises a neural interface device 402 for stimulating and/or inhibiting, preferably blocking neural activity in a nerve such as the cervical vagus nerve (not shown). Example neural interface embodiments may comprise of neural cuffs that fully or partially circumferentially enclose a segment of the nerve. The system may be used on any nerve that produces a physiological response when suitably stimulated. Examples include the cervical vagus nerve (which may produce an increase or decrease in heart rate) and the splenic nerve (which may produce an increase or decrease in blood pressure).

In other embodiments two or more neural interface devices may be provided, and any plurality of such neural interface devices may be separate or coupled. The neural interface devices may be in the form of a cuff, or any other interface suitable for attaching to or being positioned adjacent a nerve.

The neural interface device 402 comprises first, second, third and fourth electrodes 404, 405, 406, 407. Where two or more neural interface devices are provided, each may have one or more electrodes. For instance, a system may comprise first and second neural interface devices, wherein the first neural interface device comprises first and second electrodes of the system, and the second neural interface device comprises a third and fourth electrodes of the system. Alternatively, a system may comprise first, second, third and fourth neural interface devices, wherein the first neural interface device comprises a first electrode of the system, the second neural interface device comprises a second electrode of the system, the third neural interface device comprises a third electrode of the system, and the fourth neural interface device comprises a fourth electrode of the system.

The neuromodulation system 400 comprises signal generator 408 electrically coupled to the first, second, third and fourth electrodes 404, 405, 406, 407. The signal generator 408 is configured to generate a low frequency alternating current (LFAC) signal described elsewhere herein, which it applies to the nerve to which the neural interface device 402 is attached via the first, second, third and fourth electrodes 404, 405, 406, 407.

In the case of the fourth embodiment, the signal generator is configured to generate first and second LFAC electrical signals, which it applies to the nerve to which the neural interface device 402 is attached via two corresponding pairs of the first, second, third and fourth electrodes 404, 405, 406, 407. For example, the signal generator may apply the first LFAC electrical signal to the nerve by applying a signal between the first and second electrodes 404, 405 and may apply the second LFAC electrical signal to the nerve by applying a signal between the third and fourth electrodes 406, 407.

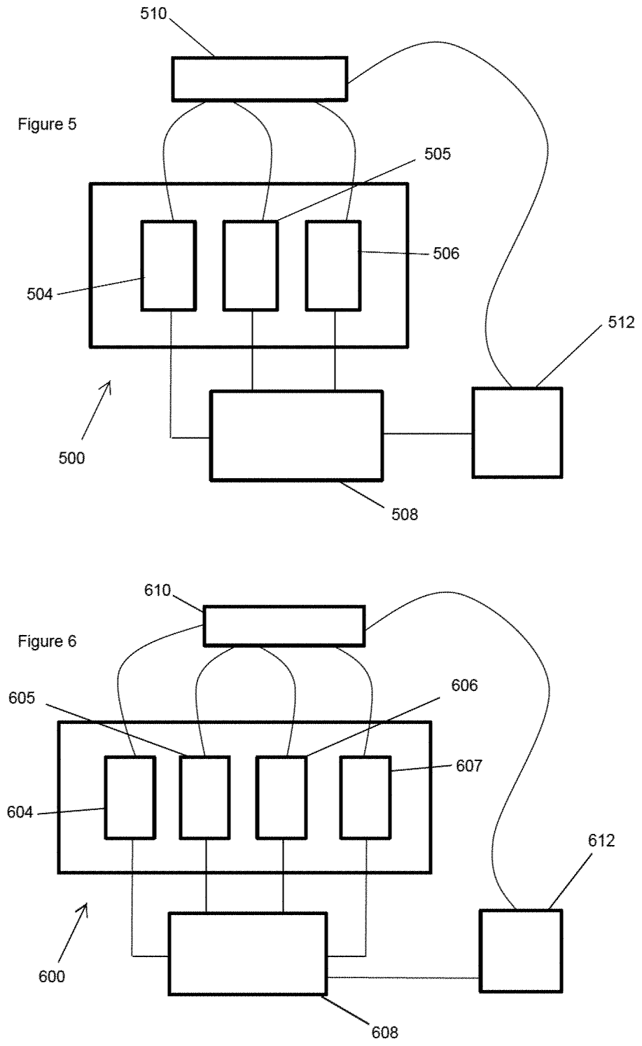

FIG. 5 shows a diagram of a fifth embodiment of an implantable neuromodulation system 500 according to the disclosure.

As shown, the embodiment shown in FIG. 5 is identical to that shown in FIG. 3, except that it comprises a voltage sensor 510 and a control system 512 as described in connection with FIG. 1.

In more detail, the neuromodulation system 500 further comprises a voltage sensor 510 coupled between the first, second and third electrodes 504, 505, 506. The voltage sensor 510 is configured to measure a voltage across the first and second electrodes 504, 505 and/or between the second and third electrodes 505, 506, and/or between the first and third electrodes 504, 506 whilst the signal generator 508 applies the LFAC signal to the nerve to which the neural interface device 502 is attached via the first, second and third electrodes 504, 505, 506.

In the case of the fifth embodiment, the voltage sensor 510 is configured to measure a voltage across two corresponding pairs of the first, second and third electrodes 504, 505, 506. For example, the voltage sensor measure a first voltage between the first and second electrodes 504, 505 and may measure a second voltage between the second and third electrodes 505, 506.

The neuromodulation system 500 further comprises a control system 512 communicatively coupled to the voltage sensor 510. The function of the control system 512 is to generate a feedback response upon receiving a signal from the voltage sensor.

FIG. 6 shows a diagram of a fifth embodiment of an implantable neuromodulation system 500 according to the disclosure.

As shown, the embodiment shown in FIG. 6 is identical to that shown in FIG. 4, except that it comprises a voltage sensor 610 and a control system 612 as described in connection with FIG. 1.

In more detail, the neuromodulation system 600 further comprises a voltage sensor 610 coupled between the first, second, third and fourth electrodes 604, 605, 606, 607. The voltage sensor 610 is configured to measure a voltage across the first and second electrodes 604, 605 and/or between the third and fourth electrodes 606, 607 whilst the signal generator 608 applies the LFAC signal to the nerve to which the neural interface device 602 is attached via the first, second, third and fourth electrodes 604, 605, 606, 607.

In the case of the sixth embodiment, the voltage sensor 610 is configured to measure a voltage across two corresponding pairs of the first, second, third and fourth electrodes 604, 605, 606, 607. For example, the voltage sensor measure a first voltage between the first and second electrodes 604, 605 and may measure a second voltage between the third and fourth electrodes 605, 606.

The neuromodulation system 600 further comprises a control system 612 communicatively coupled to the voltage sensor 610. The function of the control system 612 is to generate a feedback response upon receiving a signal from the voltage sensor.

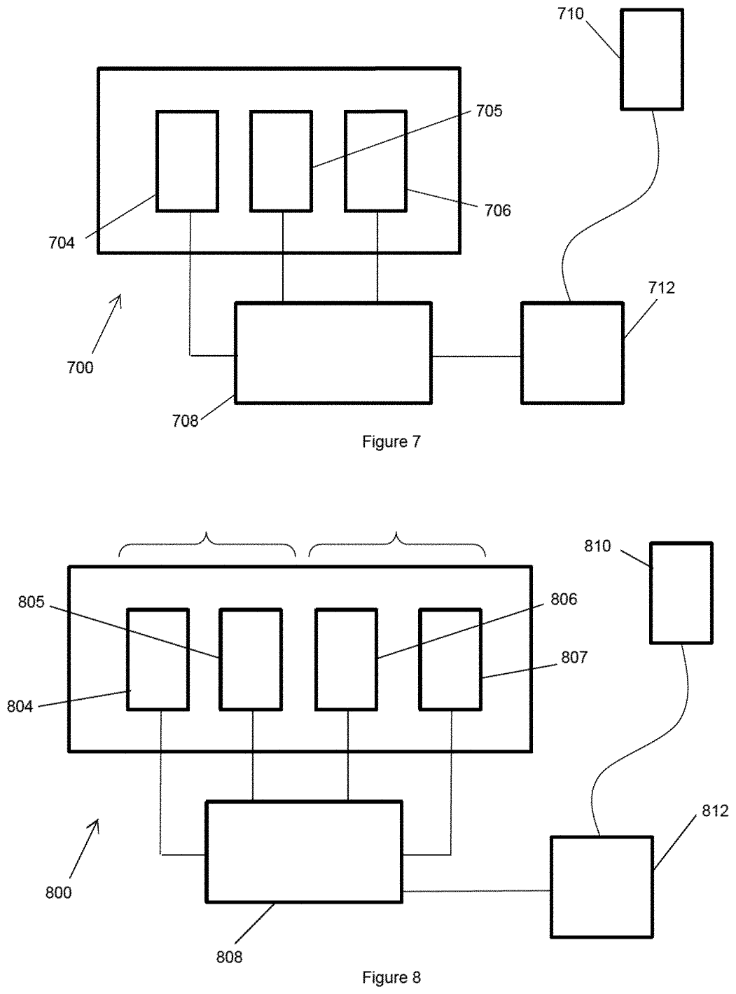

FIG. 7 shows a diagram of a seventh embodiment of an implantable neuromodulation system 700 according to the disclosure.

As shown, the embodiment shown in FIG. 7 is identical to that shown in FIG. 3, except that it comprises a physiological sensor 710 and a control system 712 as described in connection with FIG. 2.

The neuromodulation system 700 further comprises a physiological sensor 710 to detect the at least one pre-determined physiological response. In some cases, a plurality of such sensors may be used, which may sense the same or different physiological responses. Exemplary sensors include a heart rate sensor, a blood pressure sensor, a pulse oximetry sensor, a sensor for detecting cardiac function and a sensor for detecting respiratory function.

The neuromodulation system 700 further comprises a control system 712 communicatively coupled to the physiological sensor 710. The function of the control system 712 is to generate a feedback response upon receiving a signal from the physiological sensor.

FIG. 8 shows a diagram of an eighth embodiment of an implantable neuromodulation system 800 according to the disclosure.

As shown, the embodiment shown in FIG. 8 is identical to that shown in FIG. 4, except that it comprises a physiological sensor 810 and a control system 812 as described in connection with FIG. 2.

The neuromodulation system 800 further comprises a physiological sensor 810 to detect the at least one pre-determined physiological response. In some cases, a plurality of such sensors may be used, which may sense the same or different physiological responses. Exemplary sensors include a heart rate sensor, a blood pressure sensor, a pulse oximetry sensor, a sensor for detecting cardiac function and a sensor for detecting respiratory function.

The neuromodulation system 800 further comprises a control system 812 communicatively coupled to the physiological sensor 810. The function of the control system 812 is to generate a feedback response upon receiving a signal from the physiological sensor.

In all of the arrangements described above, a reference electrode (not shown) may be provided. In the case of the arrangements shown in FIGS. 1 and 2, the reference electrode is a third electrode that preferably has a larger surface area compared with the first and second electrodes 104, 106. In the case of the arrangements shown in FIGS. 3, 5 and 7, the reference electrode is a fourth electrode that preferably has a larger surface area compared with the first, second and third electrodes. In the case of the arrangements shown in FIGS. 4, 6 and 8, the reference electrode is a fifth electrode that preferably has a larger surface area compared with the first, second, third and fourth electrodes.

The reference electrode may form part, or all of a housing that encloses the implantable neuromodulation system. Alternatively, the reference electrode may be provided as a separate electrode and configured for placement away from the housing and the active electrodes (i.e. first to fourth electrodes described above).

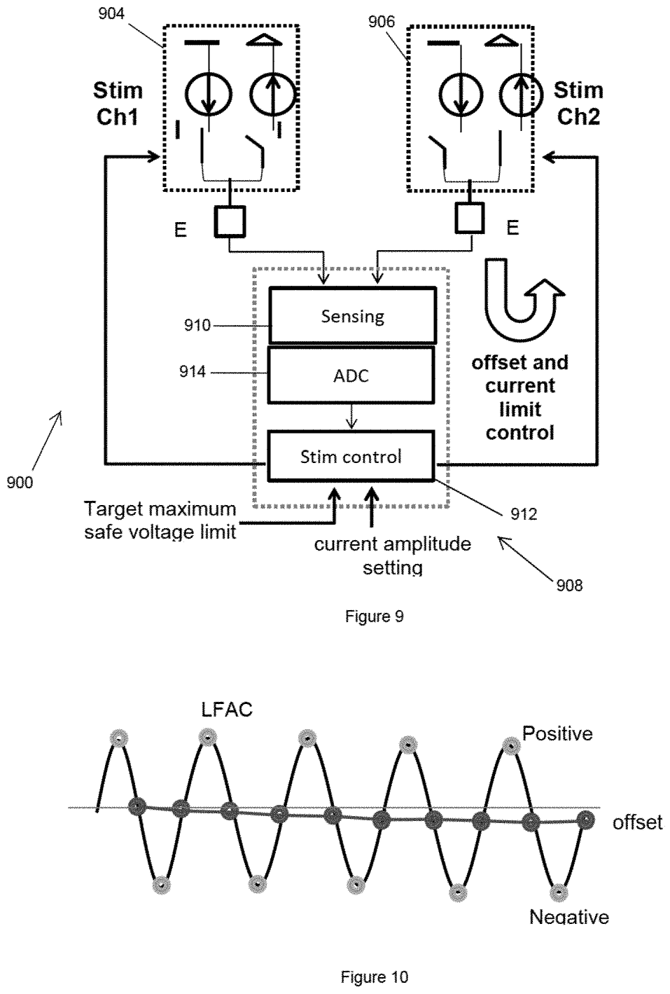

Aspects of the disclosure will now be described in connection with FIGS. 9 to 14. Together, these figures show how embodiments of the disclosure may be implemented to provide closed-loop control systems which control voltage offset of the signal across electrodes and control the magnitude of the voltage developed across the electrodes during application of a signal. In addition, the figures show how embodiments of the disclosure may be implemented to provide a closed-loop control system using disease symptoms and/or measures of physiological function including blood glucose, pulse oximetry, respiratory function, bladder pressure, and cardiovascular parameters including ECG, blood pressure, heart rhythm and rate.

FIGS. 9 and 10 show a first aspect of the disclosure. FIG. 9 shows a neuromodulation system 900 that represents an implementation of the embodiment of FIG. 1. As with FIG. 1, the system 900 of FIG. 9 comprises a neural interface device (not shown) comprising first and second electrodes 904, 906 referred to as E1 and E2 respectively. As shown diagrammatically in FIG. 9, each electrode 904, 906 comprises an arrangement whereby the electrode applies a low frequency AC (LFAC) signal that alternates between a positive current component (I_{P}) and a negative current component (I_{N}). As shown in FIG. 10, the LFAC signal has a positive voltage peak, a negative voltage peak and an offset determined by the differential voltage between the positive current component (I_{P}) and the negative current component (I_{N}).

As with FIG. 1, the neuromodulation system 900 comprises a signal generator 908 electrically coupled to the first and second electrodes 904, 906. The signal generator 908 is configured to generate a low frequency alternating current (LFAC) signal described above, which it applies to the nerve to which the neural interface device is attached via the first and second electrodes 904, 906.

The neuromodulation system 900 further comprises a voltage sensor 910 coupled between the first and second electrode 904, 906. The voltage sensor 910 is configured to measure a voltage across the first and second electrodes whilst the signal generator 908 applies the LFAC signal to the nerve to which the neural interface device is attached via the first and second electrodes 904, 906.

The neuromodulation system 900 further comprises a control system 912 communicatively coupled to the voltage sensor 910 via an analogue to digital converter (ADC) 914. Based on the measurements from the voltage sensor 910, the control system 912 determines three properties: the peak positive voltage, peak negative voltage and the voltage offset between the positive and negative current components (I_{P}) and (I_{N}).

As shown in FIG. 9, the control system 912 receives a target maximum safe voltage limit, which may for example be based upon the material of the first and second electrodes. The safe voltage limit is also known in the art as the water window limit and represents the maximum voltage which does not exceed the water window limit of the electrode surface material. For example, for platinum electrodes, the safe voltage limit is typically 1.2 v.

Based on this limit, and the measured peak positive voltage and peak negative voltage, the control system 912 can generate a control signal which causes the signal generator 908 to adjust current components (I_{P}) and (I_{N}) such that the peaks remain below the received limits. Moreover, based on the measured voltage offset, the control system 912 can generate a control signal which causes the signal generator 908 to adjust current components (I_{P}) and (I_{N}) such that the offset tends toward zero over time.

In more detail, and with reference to FIG. 10, because an LFAC is slowly varying signal, over time an offset can develop which presents the equivalent of a DC positive or negative charge (depending on the offset). It is useful to maintain an offset within the safe voltage limit (water window limit).

It will be appreciated that the peak positive voltage, peak negative voltage and the voltage offset are three examples of signal properties that may be determined by the control system 912 based on voltage measurements between the first and second electrodes 904, 906 in order for the control system 912 to generate a control signal to cause the signal generator to adjust the LFAC electrical signal to modify the respective signal property. A skilled person will appreciate that other signal properties may be determined and controlled, and accordingly the disclosure is not limited to controlling peak positive voltage, peak negative voltage or offset, though there are clear advantages not taught in the art to controlling those particular signal properties.

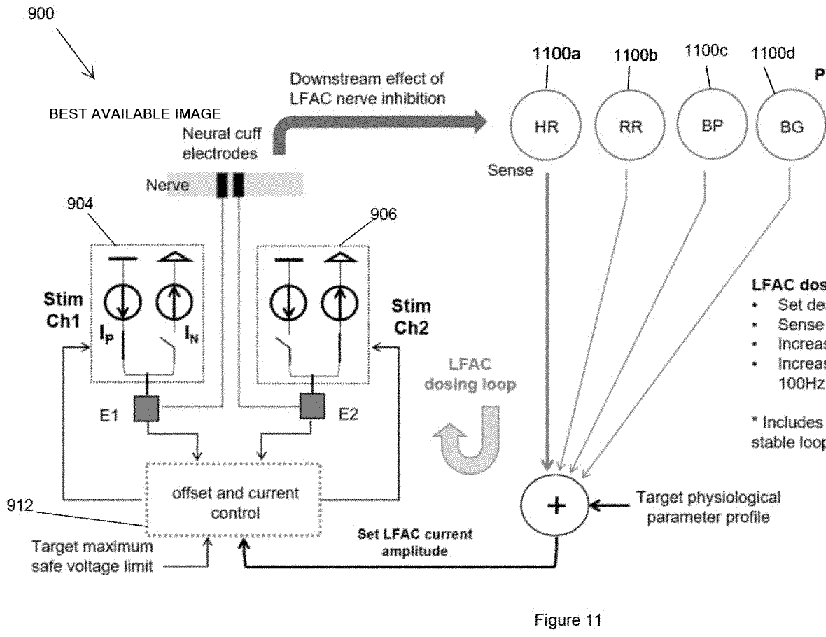

FIG. 11 shows a second aspect of the disclosure in addition to the first. FIG. 11 shows the same neuromodulation system 900 that is shown in FIG. 9 except that it comprises further components as will now be described based on FIG. 2.

The first and second electrodes 904, 906 are shown in situ attached to respective neural interface devices 902 which take the form of cuff electrodes surrounding a nerve. The application of LFAC electrical stimulation to the nerve via the first and second electrodes 904, 906 causes stimulation or inhibition (or even block) of the neural activity in the nerve, which has one or more downstream effects on physiological parameters including heart rate, pulse oximetry, respiratory rate, blood pressure and blood glucose, as well as others.

It is straightforward to configure sensors to measure a wide range of physiological parameters in a variety of settings. Indeed, sensors are known to determine heart rate, pulse oximetry, respiratory rate, blood pressure and blood glucose, and are configurable on different scales depending on the application. For instance, many sensors of physiological parameters can be implemented in wearable devices. Consumer wearable devices are commonplace which are configures to measure heart rate, pulse oximetry, respiratory rate and other parameters detectable from a contact sensor on a user's skin. Devices are also available which are capable of measuring blood pressure and blood glucose levels, for example by taking small samples of blood using microneedles. Furthermore, implantable devices are known which are configured to sense such parameters and others.

The device shown in FIG. 11 comprises four physiological sensors 1100a to 1100d which sense corresponding physiological parameters and generate corresponding signals indicative of the same. These sensors are communicatively coupled to the control system 912 in any suitable fashion including wirelessly, or via an electrical connection. The control system 912 is configured to compare the signals indicative of the four physiological parameters with a target physiological parameter profile. A target physiological parameter profile may indicate target values or target ranges for one, some, or all of the sensed physiological parameters, and based on this profile and the sensed parameters the control system 912 may determine whether one, some or all of the sensed parameters deviate from the target parameter by more than a predetermined threshold, or fall outside the respective target ranges. Based on this determination, the control system 912 can set a desired LFAC current amplitude, and communicate this value by way of a control signal to the signal generator 908 to cause it to adjust current components (I_{P}) and (I_{N}) such that the desired LFAC current amplitude is generated and transmitted to the first and second electrodes 904, 906.

It will be appreciated that the desired LFAC current amplitude may be set by determining whatever amplitude is needed to cause a block. This can be determined in a number of ways, including by intentionally stimulating and progressively increase the amplitude of the LFAC whilst measuring with another pair of electrodes to identify the point at which a block happens. Alternatively, where a block is required in order to cause or prevent a certain physiological response, then that physiological response may be measured to identify whether the LFAC current amplitude is at the correct level to achieve the occurrence of a block. In practice, a healthcare professional may set a range of current amplitude expect to be effective from experience or testing, and the current amplitude may be adjusted within that range based on physiological feedback.

It will be appreciated that heart rate, respiratory rate, blood pressure and blood glucose are four examples of physiological parameters that may be sensed and may form part of a physiological target profile such that the differential between the two can be determined by the control system 912 in order for it to generate a control signal to cause the signal generator to adjust the LFAC amplitude. A skilled person will appreciate that other physiological parameters may be sensed and utilised as part of a physiological target profile, and accordingly the invention is not limited to sensing heart rate, pulse oximetry, respiratory rate, blood pressure and blood glucose though there are clear advantages not taught in the art to sensing those particular parameters.

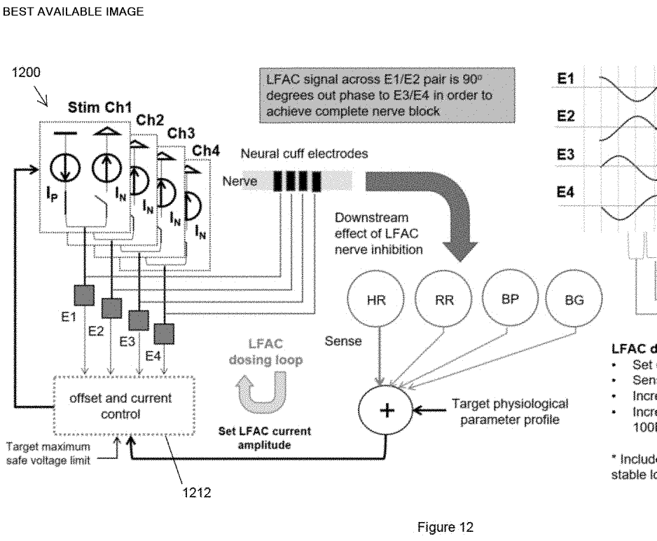

FIG. 12 shows a third aspect of the disclosure in addition to the first and second aspects described above. FIG. 12 shows a neuromodulation system 1200 that represents an implementation of the embodiments of FIGS. 4, 6 and 8. As will be seen from FIG. 12, instead of the two electrodes that are present in FIG. 11, four electrodes are provided, labelled E1, E2, E3 and E4, respectively. As with the embodiment of FIG. 11, the electrodes are shown as neural interface devices provided as cuff electrodes placed around a nerve. As with the embodiment of FIG. 11, physiological sensors are provided, which sense (for example) heart rate, pulse oximetry, respiratory rate, blood pressure and blood glucose and generate corresponding signals which may be compared with a target physiological parameter profile as described above.

A control system 1212 is provided, which provides for the same functionality as the control system described above. In particular, the control system 1212 receives a target maximum safe voltage limit upon which can be based a determination of whether the peak positive voltage or peak negative voltage of one, some or all of electrodes E1, E2, E3 and E4 exceeds a safe predetermined threshold, as described above in connection with FIGS. 9 and 10 (and described elsewhere herein). Moreover, the control system 1212 receives a target physiological parameter profile upon which can be based a determination of a suitable LFAC amplitude, as described above in connection with FIG. 11.

The four electrodes E1, E2, E3 and E4 are configured to operate, in conjunction with the signal generator (not shown), in two pairs. In particular, in the embodiment of FIG. 12, electrodes E1 and E2 are configured to operate as one pair, whilst E3 and E4 are configured to operate as a second pair. As would be expected when an LFAC signal passes from one electrode of the pair to the other, it can be seen from the graphs shown in FIG. 12 that the current measured at one of the electrodes is the opposite polarity (but equal magnitude) of the current at the other of the electrodes. In other words, both electrodes in a pair experience zero current draw at the same points in time, and when one of the electrodes experiences peak positive current, the other experiences peak negative current.

Moreover, the current draw (i.e. signal) through the first pair of electrodes E1 and E2 is 90° out of phase with the current draw through the second pair of electrodes E3 and E4. 20 This achieves complete nerve block by maintaining a peak positive (or peak negative) current draw through one of the electrodes every 90°. As shown in the graphs on FIG. 12, over time the electrode pair blocking sequence is E2/E1, followed by E4/E3, followed by E1/E2, followed by E3/E4, and repeat. In other words, over a complete cycle the peak positive current occurs at E2, E4, E1 and E3, changing every 90°.

It will of course be appreciated that the principle of this aspect of the disclosure can be applied to configurations with any number of electrodes. For instance, an arrangement comprising 6 electrodes formed of three pairs may be arranged such that the signal through each pair of electrodes is 60° out of phase with another pair.

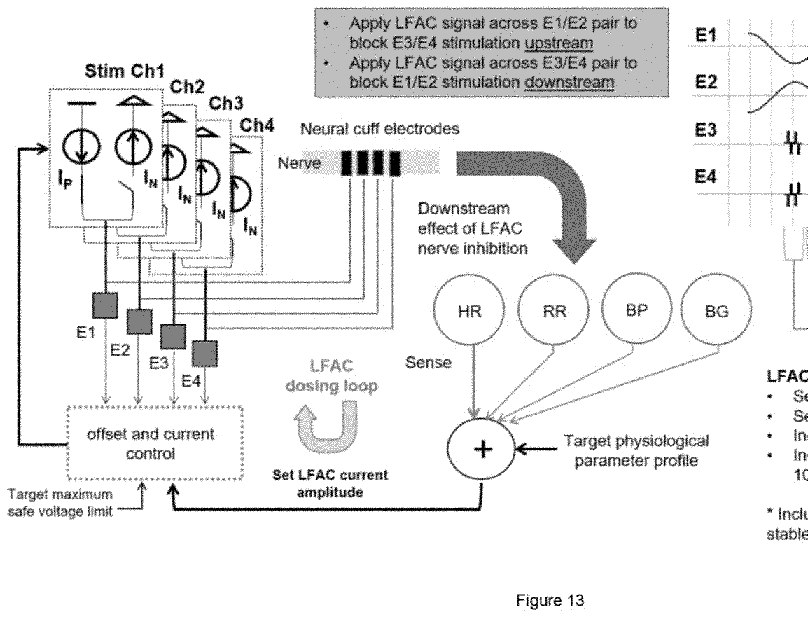

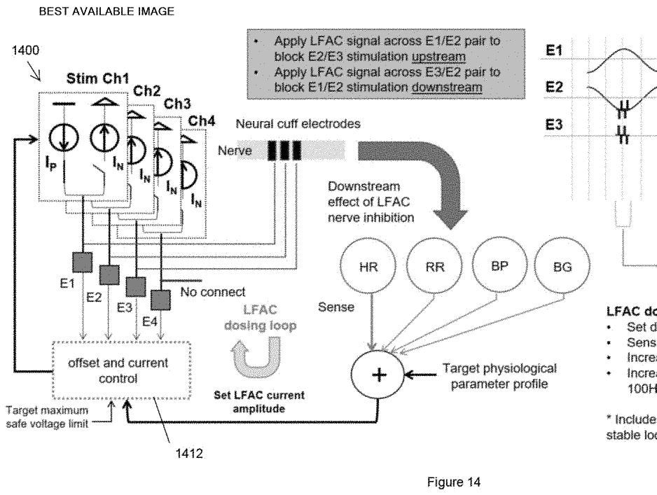

FIGS. 13 and 14 show a fourth aspect of the disclosure in addition to the first to third aspects described above. FIG. 13 shows an identical configuration to that shown in FIG. 12, whereby the four electrodes E1, E2, E3 and E4 are provided in two pairs; namely a first pair comprising electrodes E1 and E2, and a second pair comprising electrodes E3 and E4. Whereas the arrangement shown in FIG. 12 applies two out-of-phase LFAC signals to the first and second pairs of electrodes in order to achieve complete block, the arrangement shown in FIG. 13 applies one LFAC signal to the first pair of electrodes and a stimulation signal to the second pair of electrodes so as to achieve directional stimulation by virtue of the LFAC signal blocking the stimulation signal in a particular direction.

In more detail, where FIG. 13 differs from FIG. 12 is that an LFAC signal is applied across one of the pairs of electrodes (in this example, the first pair of electrodes E1 and E2) in order to block a stimulation signal applied to the other of the pair of electrodes (in this example, the second pair of electrodes E3 and E4) either upstream or downstream depending on which pair is used for which signal. For example, as shown, an LFAC signal may be applied across the first pair of electrodes E1 and E2, so as to block a stimulation signal from the second pair of electrodes E3 and E4 upstream. Conversely, an LFAC signal may be applied across the second pair of electrodes E3 and E4, so as to block a stimulation signal from the first pair of electrodes E1 and E2 downstream. In connection with this arrangement, it is relevant that the first pair of electrodes is upstream of the second pair of electrodes.

It will be noted that the signal generator is configured to time the application of the stimulation signal with a peak and/or a trough in the LFAC signal, which is when the LFAC signal is configured to block propagation of nerve signals. For example, as shown in FIG. 13, a stimulation signal is applied to the nerve via E3 and E4 every 90° phase of the LFAC signal, when the LFAC signal is at a peak at either the E1 or E2. Since E1 and E2 are upstream of E3 and E4 (in this example), the illustrated arrangement will block the stimulation signal from propagating upstream. Were E1 and E2 to be downstream of E3 and E4 (or were the LFAC signal to be applied to E3 and E4 and the stimulation signal to be applied to E1 and E2), the arrangement will block the stimulation signal from propagating downstream.

Finally, FIG. 14 shows a variation of a neuromodulation system 1400 that represents an implementation of the embodiments of FIGS. 3, 5 and 7. As will be seen from FIG. 14, instead of the two electrodes that are present in FIG. 11 (and the four electrodes that are present in FIGS. 12 and 13, three electrodes are provided, labelled E1, E2 and E3, respectively. As with the embodiments of FIGS. 11 to 13, the electrodes are shown as neural interface devices provided as cuff electrodes placed around a nerve. As with the embodiments of FIGS. 11 to 13, physiological sensors are provided, which sense (for example) heart rate, respiratory rate, pulse oximetry, blood pressure, ECG, bladder pressure and blood glucose and generate corresponding signals which may be compared with a target physiological parameter profile as described above.

A control system 1412 is provided, which provides for the same functionality as the control system described above. In particular, the control system 1412 receives a target maximum safe voltage limit upon which can be based a determination of whether the peak positive voltage or peak negative voltage of one, some or all of electrodes E1, E2 and E3 exceeds a safe predetermined threshold, as described above in connection with FIGS. 9 and 10 (and described elsewhere herein). Moreover, the control system 1412 receives a target physiological parameter profile upon which can be based a determination of a suitable LFAC amplitude, as described above in connection with FIG. 11.

The three electrodes E1, E2 and E3 are configured to operate, in conjunction with the signal generator (not shown), in two pairs. In particular, in the embodiment of FIG. 14, electrodes E1 and E2 are configured to operate as one pair, whilst E2 and E3 are configured to operate as a second pair. As would be expected when an LFAC signal passes from one electrode of the pair to the other, it can be seen from the graphs shown in FIG. 12 that the current measured at one of the electrodes is the opposite polarity (but equal magnitude) of the current at the other of the electrodes. In other words, both electrodes in a pair experience zero current draw at the same points in time, and when one of the electrodes experiences peak positive current, the other experiences peak negative current.

As with the arrangement shown in FIG. 13, the arrangement shown in FIG. 14 applies one LFAC signal to the first pair of electrodes and a stimulation signal to the second pair of electrodes so as to achieve directional stimulation by virtue of the LFAC signal blocking the stimulation signal in a particular direction. It will be appreciated that the signal applied to E2 is a composite signal made up of the LFAC signal applied to the first pair of electrodes (E1 and E2) plus the stimulation signal applied to the second pair of electrodes (E2 and E3)

In more detail, as with FIG. 13, an LFAC signal is applied across one of the pairs of electrodes (in this example, the first pair of electrodes E1 and E2) in order to block stimulation from the other of the pairs of electrodes (in this example, the second pair of electrodes E3 and E4) either upstream or downstream depending on which pair is used for which signal. For example, as shown, an LFAC signal may be applied across the first pair of electrodes E1 and E2, so as to block stimulation from the second pair of electrodes E2 and E3 upstream. Conversely, an LFAC signal may be applied across the second pair of electrodes E2 and E3, so as to block stimulation from the first pair of electrodes E1 and E2 downstream. In connection with this arrangement, it is relevant that the first pair of electrodes is upstream of the second pair of electrodes (or, in particular, the first electrode is upstream of the second electrode, which is upstream of the third electrode).

It will be noted that the signal generator is configured to time the application of the stimulation signal with a peak and/or a trough in the LFAC signal, which is when the LFAC signal is configured to block propagation of nerve signals. For example, as shown in FIG. 14, a stimulation signal is applied to the nerve via E2 and E3 every 180° phase of the LFAC signal, when the LFAC signal is at a peak at E1 and a trough at E2. Since E1 is upstream of E3 (in this example), the illustrated arrangement will block the stimulation signal from propagating upstream. Were E1 to be downstream of E3 (or were the LFAC signal to be applied to E2 and E3 and the stimulation signal to be applied to E1 and E2), the arrangement will block the stimulation signal from propagating downstream.

Although the examples given above utilize a sinusoidal signal as the LFAC signal, it need not be perfectly sinusoidal. Signals approximating a sinusoidal shape, as well as triangular or saw-toothed signals may be utilized instead.

It will be appreciated that the disclosure has been described in connection with preferred embodiments, but modifications can be made to those embodiments without departing from the scope of the invention as defined by the appended claims.

1-34. (canceled)

35. An implantable neuromodulation system for delivering an electrical signal, preferably an LFAC electrical signal, to a nerve to stimulate or inhibit, optionally block, conduction of action potentials in the nerve, the system comprising:
a neural interface device for stimulating or inhibiting, optionally blocking, neural activity in the nerve, the at least one neural interface device comprising first and second electrodes;
a signal generator electrically coupled to the first and second electrodes and configured to generate the electrical signal that, when applied to the nerve via the first and second electrodes, stimulates or inhibits, optionally blocks, neural activity in the nerve;
a first closed-loop controller comprising a sensor coupled between the first and second electrodes and configured to determine a voltage across the first and second electrodes when the electrical signal is applied to the nerve via the first and second electrodes, the first closed-loop controller further comprising a processor to determine a property of the signal based on the measured voltage;
wherein the first closed-loop controller is further configured to generate a control signal based on the determined property, the control signal configured to cause the signal generator to adjust the electrical signal to modify the property of the signal.

36. The system of claim 35, wherein the determined property is a voltage offset between the first and second electrodes, and wherein the control signal is configured to cause the signal generator to adjust the electrical signal to reduce the voltage offset, preferably toward 0 v, more preferably to 0 v.

37. The system of claim 35, wherein the determined property is a magnitude of the voltage across the first and second electrodes, and wherein the control signal is configured to cause the signal generator to adjust the electrical signal to reduce the magnitude of the voltage.

38. The system of claim 37, wherein the processor is further configured to compare the determined magnitude of the voltage across the first and second electrodes with a predetermined threshold, and generate the control signal based on the comparison.

39. The system of claim 38, wherein the predetermined threshold is an upper voltage limit, preferably a safe voltage limit, more preferably a safe voltage limit based upon the material of the first and second electrodes.

40. The system of claim 35, further comprising a second closed-loop controller comprising a physiological sensor configured to measure a physiological parameter of a patient when the electrical signal is applied to the nerve via the first and second electrodes, wherein the physiological parameter is affected by the stimulation or inhibition, optionally block, of the neural activity in the nerve, the second closed-loop controller further comprising a processor to determine a deviation from a target physiological parameter, based on the measured physiological parameter;
wherein the second closed-loop controller is further configured to generate a control signal based on the deviation, the control signal configured to cause the signal generator to adjust the electrical signal to reduce the deviation from the target physiological parameter.

41. The system of claim 35, wherein the physiological sensor is one or more of a heart rate sensor, respiratory rate sensor, pulse oximetry sensor, blood pressure sensor and blood glucose sensor, and physiological parameter is one or more of heart rate, respiratory rate, pulse oximetry, blood pressure and blood glucose.

42. The system of claim 35, wherein the system comprises a third electrode, preferably having a larger surface area than the first and second electrodes.

43. An implantable neuromodulation system for delivering electrical signals, preferably LFAC electrical signals, to a nerve to stimulate or inhibit, optionally block, conduction of action potentials in the nerve, the system comprising:
a neural interface device for stimulating or inhibiting, optionally blocking, neural activity in the nerve, the at least one neural interface device comprising at least first and second pairs of electrodes;
a signal generator electrically coupled to the at least first and second pairs of electrodes and configured to generate first and second electrical signals that, when applied to the nerve via the first and second pairs of electrodes respectively, stimulates or inhibits, optionally blocks, neural activity in the nerve;
wherein the first electrical signal applied to the first pair of electrodes is out of phase with the second electrical signal applied to the second pair of electrodes.

44. The system of claim 43, wherein the first pair of electrodes comprises first and second electrodes, and the second pair of electrodes comprises third and fourth electrodes, further wherein the first and second electrical signals are configured such that, when applied to the nerve via the first and second pairs of electrodes respectively, the first electrical signal stimulates or inhibits, optionally blocks, neural activity in the nerve for a first period and the second electrical signal stimulates or inhibits, optionally blocks, neural activity in the nerve for a second period following the first period, further wherein the first and second electrical signals are further configured such that, when applied to the nerve via the first and second pairs of electrodes respectively, the first electrical signal stimulates or inhibits, optionally blocks, neural activity in the nerve for a third period following the second period, and the second electrical signal stimulates or inhibits, optionally blocks, neural activity in the nerve for a fourth period following the third period, further wherein each period corresponds to a 90° phase of the first and second electrical signals.

45. The system of claim 43, wherein the first and second electrical signals are configured such that, when applied to the nerve via the first and second pairs of electrodes respectively, the first electrical signal stimulates or inhibits, optionally blocks, neural activity in the nerve in an upstream direction from stimulation by the second electrical signal, and the second electrical signal stimulates or inhibits, optionally blocks, neural activity in the nerve in a downstream direction from stimulation by the first electrical signal.

46. The system of claim 43, wherein the first electrical signal is out of phase with the second electrical signal by between 1° and 180°, preferably by between 45° and 135°, more by preferably by between 80° and 100°, most preferably by 90°.

47. The system of claim 43, further comprising:
a physiological closed-loop controller comprising a physiological sensor configured to measure a physiological parameter of a patient when the electrical signal is applied to the nerve via the first and second electrodes, wherein the physiological parameter is affected by the stimulation or inhibition, optionally blocking, of the neural activity in the nerve, the physiological closed-loop controller further comprising a processor to determine a deviation from a target physiological parameter, based on the measured physiological parameter;
wherein the physiological closed-loop controller is further configured to generate a control signal based on the determined deviation, the control signal configured to cause the signal generator to adjust the electrical signal to reduce the deviation from the target physiological parameter.

48. The system of claim 43, further comprising:
a signal property closed-loop controller comprising a sensor coupled between the first and second electrodes and configured to determine a voltage across the first and second electrodes when the electrical signal is applied to the nerve via the first and second electrodes, the signal property closed-loop controller further comprising a processor to determine a property of the signal based on the measured voltage;
wherein the signal property closed-loop controller is further configured to generate a control signal based on the determined property, the control signal configured to cause the signal generator to adjust the electrical signal to modify the property of the signal.

49. An implantable neuromodulation system for delivering electrical signals to a nerve to stimulate or inhibit, optionally block, conduction of action potentials in the nerve, the system comprising:
a neural interface device for stimulating or inhibiting, optionally blocking, neural activity in the nerve, the at least one neural interface device comprising at least first and second pairs of electrodes;
a signal generator electrically coupled to the at least first and second pairs of electrodes and configured to generate an inhibition electrical signal, preferably an LFAC electrical signal, that, when applied to the nerve via the first pair of the electrodes, inhibits, optionally blocks, neural activity in the nerve; the signal generator further configured to generate a stimulation signal that, when applied to the nerve via the second pair of the electrodes, stimulates neural activity in the nerve;
wherein the signal generator is configured to apply the stimulation signal to the nerve via the second pair of electrodes during a blocking window of the inhibition electrical signal applied to the nerve via the first pair of electrodes such that the inhibition electrical signal applied to the nerve via the first pair of electrodes inhibits, optionally blocks, propagation of the stimulation signal applied to the nerve via the second pair of electrodes.

50. The system of claim 49, wherein the blocking window is within a predetermined threshold phase difference from a peak and/or trough of the inhibition electrical signal, further wherein the predetermined threshold phase difference is ±90°, preferably ±70°, preferably ±55°, preferably ±45°, preferably ±35°, preferably ±25°, preferably ±15°, preferably ±10°, preferably ±5°, preferably ±2°, preferably ±1° of the peak and/or trough of the inhibition electrical signal.

51. The system of claim 49, wherein the neural interface device comprises at least first, second and third electrodes, and wherein the first pair of electrodes comprises the first and second electrodes, and the second pair of electrodes comprises the second and third electrodes,
further wherein the neural interface device is configured in use to extend along a nerve from a first end of the device to a second end, and wherein the first and second pairs of electrodes are spaced apart between the first and second ends such that at least one electrode of the first pair of electrodes is proximate the first end and at least one electrode of the second pair of electrodes is proximate the second end,
further wherein the first electrode is proximate the first end, the third electrode is proximate the second end and the second electrode is between the first and third electrodes,
further wherein the signal generator is configured to apply the stimulation signal to the nerve via the second pair of electrodes when the inhibition electrical signal applied to the nerve via the first pair of electrodes is within a predetermined threshold phase difference from a trough of the LFAC electrical signal at the second electrode.

52. The system of claim 49, wherein the neural interface device comprises at least first, second, third and fourth electrodes, and wherein the first pair of electrodes comprises the first and second electrodes, and the second pair of electrodes comprises the third and fourth electrodes,
further wherein the neural interface device is configured in use to extend along a nerve from a first end of the device to a second end, and wherein the first and second pairs of electrodes are spaced apart between the first and second ends such that at least one electrode of the first pair of electrodes is proximate the first end and at least one electrode of the second pair of electrodes is proximate the second end,
further wherein the first and second electrodes are proximate the first end, and the third and fourth electrodes are proximate the second end.

53. The system of claim 49, further comprising:
a physiological closed-loop controller comprising a physiological sensor configured to measure a physiological parameter of a patient when the electrical signal is applied to the nerve via the first and second electrodes, wherein the physiological parameter is affected by the stimulation or inhibition, optionally blocking, of the neural activity in the nerve, the physiological closed-loop controller further comprising a processor to determine a deviation from a target physiological parameter, based on the measured physiological parameter;
wherein the physiological closed-loop controller is further configured to generate a control signal based on the determined deviation, the control signal configured to cause the signal generator to adjust the electrical signal to reduce the deviation from the target physiological parameter.

54. The system of claim 49, further comprising:
a signal property closed-loop controller comprising a sensor coupled between the first and second electrodes and configured to determine a voltage across the first and second electrodes when the electrical signal is applied to the nerve via the first and second electrodes, the signal property closed-loop controller further comprising a processor to determine a property of the signal based on the measured voltage;
wherein the signal property closed-loop controller is further configured to generate a control signal based on the determined property, the control signal configured to cause the signal generator to adjust the electrical signal to modify the property of the signal.
